(19) United States
(12) Patent Application Publication
(10) Pub. No.: US 2019/0307170 A1
(43) Pub. Date: Oct. 10, 2019

(54) Systems and Methods to Precisely Vaporize Liquid Chemicals

(71) Applicants: Pezhman Zarifian, Van Nuys, CA (US); Camelia C. Sehat, Van Nuys, CA (US)

(72) Inventors: Pezhman Zarifian, Van Nuys, CA (US); Camelia C. Sehat, Van Nuys, CA (US)

(21) Appl. No.: 16/378,410

(22) Filed: Apr. 8, 2019

Related U.S. Application Data
(60) Provisional application No. 62/655,655, filed on Apr. 10, 2018.

(51) Int. Cl. A24F 47/00; A24B 15/16; A61M 11/00; A61M 11/04; G05B 19/042; G05D 11/13

(52) U.S. Cl. CPC A24F 47/008; A24B 15/167; A61M 11/005; H02J 7/0063; A61M 11/007; G05B 19/042; G05D 11/132; A61M 11/041

(57) ABSTRACT

Systems and methods of vaporizing liquids are disclosed. In some embodiments, acoustic sources are implemented to motivate fluids out of liquid-containing cartridges in precise quantities such that the fluids can be combined in a desired ratio for later vaporization and inhalation by a user. A liquid cartridge can include a resonance chamber to hold the liquid, where the resonance chamber improves an acoustic source's ability to cause fluid to leave the liquid cartridge when it is vibrated at its resonance frequency. It is also contemplated that systems of the inventive subject matter can include pressure-generating pumps to motivate fluid to leave the liquid cartridges. Liquid leaving cartridges passes into microfluidic channels and later combined before being vaporized for inhalation.

This application claims priority to U.S. provisional patent application, Ser. No. 62/655655, filed Apr. 10, 2018. All extrinsic materials identified in this application are incorporated by reference in their entirety.

### FIELD OF THE INVENTION

The field of the invention is vaporization of liquid chemicals for inhalation.

### BACKGROUND

The background description includes information that may be useful in understanding the present invention. It is not an admission that any of the information provided in this application is prior art or relevant to the presently claimed invention, or that any publication specifically or implicitly referenced is prior art.

Vaporization of liquid chemicals is a growing industry, driven by both recreational and medical users. Liquids can be vaporized to deliver various flavors and chemicals, including nicotine. With legalization of medical and recreational use of marijuana in many states around the country, liquid vaporization devices are increasingly designed to administer specific types and quantities of chemicals typically found in marijuana.

Innovations and improvements have been made in this field over the years. For example, U.S. Patent Publication No. 2016/0331022 describes the need for a device that can mix quantities of tetrahydrocannabinol (THC), cannabidiol (CBD), and cannabinol (CBN). The application describes a physical device that that can create such mixtures, but it fails to appreciate technological improvements that can improve the ability of vaporization devices to administer more precise doses and quantities of various chemicals.

In U.S. Patent Publication No. 2015/0223523, the inventors tried to improve vaporization devices by describing systems and methods of depositing chemicals onto a substrate for later vaporization and inhalation. But this application fails to appreciate that improvements in microfluidics facilitate the creation of more precise mixtures of liquid chemicals for vaporization and subsequent inhalation using a single delivery device and one or more cartridges containing liquid chemicals.

These and all other extrinsic materials discussed in this application are incorporated by reference in their entirety. Where a definition or use of a term in an incorporated reference is inconsistent or contrary to the definition of that term provided in this application, the definition of that term provided in this application applies and the definition of that term in the reference does not apply.

It has yet to be appreciated that microfluidics can be implemented into electronic vaporizers to create precise mixtures of liquid chemicals for vaporization and inhalation. Thus, there is still a need in the art for improved vaporizers for medical and recreational inhalation of vaporized liquids.

### SUMMARY OF THE INVENTION

The present invention provides apparatus, systems, and methods directed to vaporization of liquids for inhalation. In one aspect of the inventive subject matter, a vaporizer is contemplated that includes: a first cartridge connector configured to receive a first cartridge containing a first vaping liquid and configured to vaporize the first vaping liquid upon activation; a second cartridge connector configured to receive a first cartridge containing a second vaping liquid and configured to vaporize the second vaping liquid upon activation; a microcontroller configured to create a blend of vaporized vaping liquid, the blend comprising the first vaping liquid and the second vaping liquid, wherein the blend comprises a pre-specified ratio of the first vaping liquid to the second vaping liquid. Upon coupling the first and second cartridges to the first and second cartridge connectors, the microcontroller is configured to activate the first cartridge to vaporize the first vaping liquid for a first duration of time and to activate the second cartridge to vaporize the second vaping liquid for a second duration of time. In some embodiments, the first duration of time is different from the second duration of time, and the pre-specified ratio of the blend is correlated with a ratio of the first duration of time to the second duration of time.

In some embodiments, the ratio of the first duration of time to the second duration of time is approximately the same as the pre-specified ratio. Some embodiments of the device also include a display and at least one input, which can be used, for example, to give a user information about a vaporizer's current settings and to allow a user to change those settings.

In some embodiments, vaporizers include a wireless communication module to facilitate connection a mobile device. The mobile device can be configured to receive input from a user and transmit information based on the input to the vaporizer, the input comprising settings for the vaporizer. It is contemplated that a vaporizer's settings can include one or both of a first time duration and a second time duration.

In some embodiments, the microcontroller is configured to activate the first cartridge using a pulse-width modulator that generates electrical pulses according to a duty cycle and a switching frequency. The duty cycle results in the first cartridge being activated for the first duration of time, and wherein the first duration of time is distributed over the second duration of time according to the duty cycle and the switching frequency.

In some embodiments, the microcontroller in a vaporizer can additionally be configured to activate the first cartridge using a first pulse-width modulator that generates electrical pulses according to a first duty cycle and a first switching frequency and to activate the second cartridge using a second pulse-width modulator that generates electrical pulses according to a second duty cycle and a second switching frequency. In such embodiments, the first and second cartridges are activated over a total time duration, where the total time duration is longer than both the first time duration and the second time duration (individually or combined). In some embodiments, the first duty cycle is different from the second duty cycle, and it is also contemplated that the first cartridge and the second cartridge can be activated for at least some overlapping amount of time.

One should appreciate that the disclosed subject matter provides many advantageous technical effects including an ability to create custom blends of two or more vaping liquids, where the ratio of each liquid can be set according to a pre-specified ratio.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic of an embodiment of a vaporization device incorporating acoustic actuators.

FIG. 2 is a schematic of an embodiment of a vaporization device incorporating pump actuators.

FIG. 3 shows microplexed droplet actuation using an acoustic source.

FIG. 4 shows how four liquids from microfluidic channels can be merged into a single channel.

FIG. 5 shows a schematic of another embodiment of a vaporizer.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used in the description in this application and throughout the claims that follow, the meaning of “a,” “an,” and “the” includes plural reference unless the context clearly dictates otherwise. Also, as used in the description in this application, the meaning of “in” includes “in” and “on” unless the context clearly dictates otherwise.

Also, as used in this application, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth in this application should be interpreted as being inclusive of their endpoints and open-ended ranges should be interpreted to include only commercially practical values. Similarly, all lists of values should be considered as inclusive of intermediate values unless the context indicates the contrary.

It should be noted that any language directed to a computer should be read to include any suitable combination of computing devices, including servers, interfaces, systems, databases, agents, peers, Engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network. The following description includes information that may be useful in understanding the present invention. It is not an admission that any of the information provided in this application is prior art or relevant to the presently claimed invention, or that any publication specifically or implicitly referenced is prior art.

Systems and methods of the inventive subject matter are directed to precision-controlled single- or multi-cartridge vaporizers that allow users to precisely control a ratio of two or more vaping liquids based on personal preference. There currently exists no smoking or vaping device that can control the delivery of a precise dosage and mix of chemicals (e.g., cannabis related chemicals) to a user at a particular temperature. The inventive subject matter is directed to electronic vaporizers (e.g., e-cigarettes and vapes) that can be loaded with liquid cannabis oil cartridges (e.g., cartridges containing one or more of tetrahydrocannabinol (THC), cannabidiol (CBD), and cannabinol (CBN)) of various strains. Vaporizers of the inventive subject matter allow users to precisely control dosage, mix of strains, mix of chemical compounds, vapor temperature. etc. to control the effect of cannabis consumption and smoking experience.

Systems of the inventive subject matter can mix any type of liquids capable of vaporization and controlling the ratio of those liquids can be useful in a wide variety of different applications. For example, systems of the inventive subject matter can help people break nicotine addiction. Vaping liquid(s) having nicotine can be mixed with nicotine-free vaping liquids at gradually changing ratios until the nicotine liquid is phased out entirely. This method can help a nicotine user quit by gradually titrating down the amount of nicotine consumed by the user in a given time period as opposed to abruptly dropping the nicotine level in their system as is done with other methods (e.g., nicotine patches).

It is contemplated that systems, devices, and methods of the inventive subject matter can be used in medical and recreational situations. In a medical context, a patient can use systems and methods of the inventive subject matter to create customized vaporized liquid mixtures and doses (e.g., liquids containing THC, CBD, and CBN) for inhalation to treat ailments (e.g., chronic pain, insomnia, and glaucoma). In a recreational context, systems, devices, and methods of the inventive subject matter can be used to facilitate recreational inhalation of customized quantities and ratios of vaporized liquid chemicals including THC, CBD, and CBN. Chemicals to be vaporized and inhaled can be contained in liquid oils that are stored in cartridges.

Determining recommended quantities and mixtures of substances contained within liquid cartridges can be done using a computing device having executable code configured to make such a determination using information about a particular user or patient. Users of systems and methods of the inventive subject matter can also adjust dosing, quantities, and mixtures of vaporized liquids according to personal preference. For example, a system could be used with two cartridges, one having indica-dominant liquid, one having sativa-dominant liquid, and the user can determine the quantities and ratios of each to inhale during each use of the system, where a “use” in this context is an action where the liquids are vaporized for inhalation.

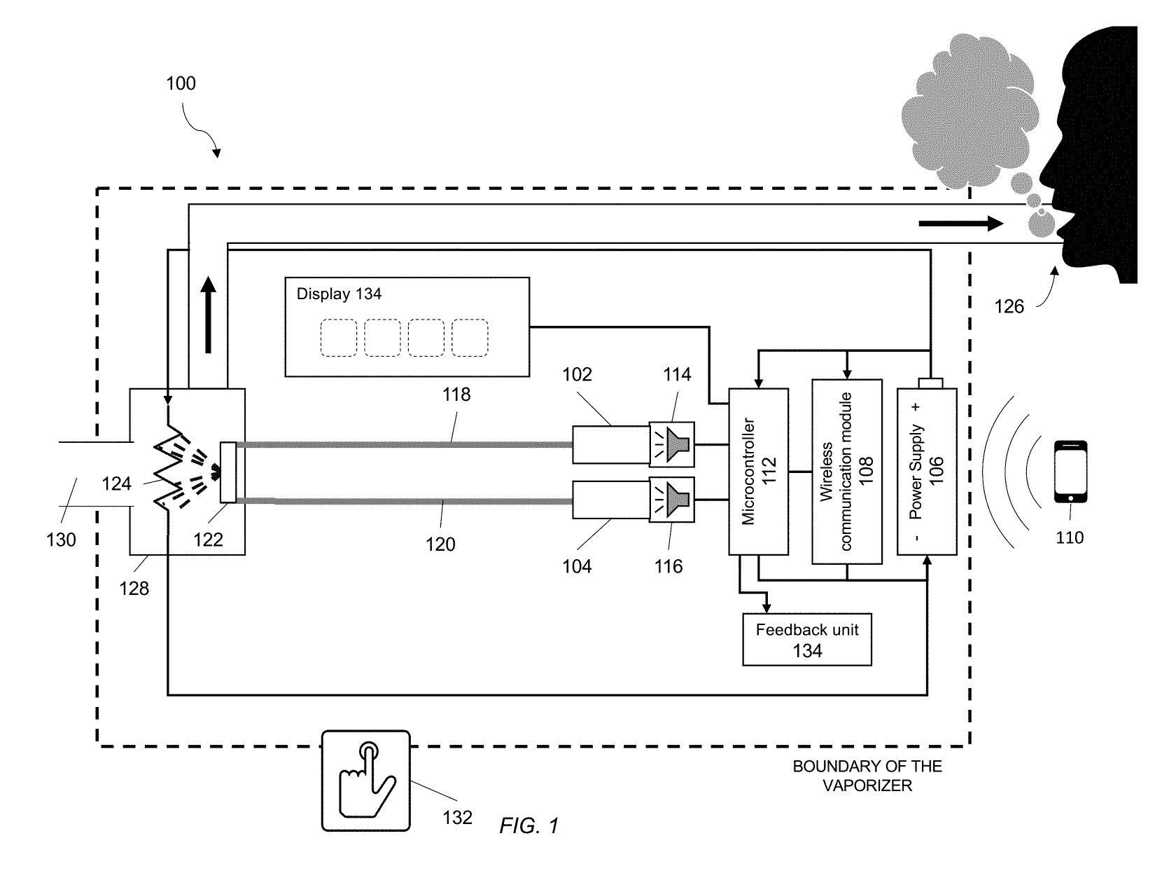

FIG. 1 shows a vaporizer 100 of the inventive subject matter that accommodates two liquid cartridges 102 & 104. It includes a power source 106, which can be, for example, a rechargeable battery or any other suitable power supply, whether internal or external. A power source is coupled, either directly or indirectly, with all electronic components of systems of the inventive subject matter. Some embodiments include a wireless communication module 108 (e.g., a Bluetooth or WiFi module). The wireless communication module 108 can facilitate communications with an external computing device 110 (e.g., a mobile phone or other computing device that can communicate via wireless connection).

Including a wireless communication module 108 can make it possible to change settings on a vaporizer 100 without directly manipulating the vaporizer 100 itself. In some embodiments, the vaporizer 100 can receive instructions about quantities and mixture ratios remotely and adjust the vaporizer's function accordingly. For example, the microcontroller 112 can adjust the outputs of the actuators 102 & 104 can have their outputs modulated according to a desired fluid flow through the microfluidic channels).

A microcontroller 112 can also be included. The microcontroller 112 is responsible for operating the vaporizer 100. Operating parameters for a vaporizer 100 can be user-defined, factory-defined, automatically defined (e.g., by software), or any combination thereof. In embodiments where operating parameters are automatically defined, it is contemplated that computer software that develops the parameters can account for personal information about a user (e.g., medical information, lifestyle information, etc.). In some embodiments, the parameters are defined externally from the vaporizer (e.g., on a computing device with companion software), while in others, the set of parameters can be input directly to a vaporizer. Operating parameters, as well as other executable code necessary for the device to function, can be stored on memory that is either internal or external to the vaporizer 100 so long as the parameters can be implemented by the microcontroller 112.

It is contemplated that electrical, mechanical, and electromechanical components of vaporizers of the inventive subject matter can be operated by the microcontroller. For example, if a user sets a parameter that calls for 30% of a fluid from one cartridge and 70% from another cartridge, then the microcontroller 112 can transmit signals to actuators 114 & 116. It is contemplated that actuators 114 & 116 can be configured to generate mechanical energy in the form of compression waves (e.g., sound) at a variety of different frequencies and amplitudes. Piezoelectric transducers or miniature speakers of any kind can be implemented. Those signals can then cause actuators 114 & 116 to emit mechanical energy (e.g., compression waves), and that mechanical energy causes fluid to flow out of cartridges 114 & 116 and into microfluidic channels 118 & 120 according to, for example, a desired ratio of liquids, a desired volume of liquids, or both.

When actuators 114 & 116 cause liquid from the cartridges 102 & 104 to move into the microfluidic channel 118 & 120, the liquids move toward a combining chamber 122. The liquids are combined within the mixing chamber and then sprayed out (e.g., via an atomizer) to be vaporized by a heating element 124. The heating element 124 is operated by the microcontroller, which can affect temperature, duration of energization, and so on. It is contemplated that the combining chamber 122 can include an atomizer for each vaping liquid coming from the different cartridges 102 & 104. In these embodiments, the combining chamber 122 causes the liquids to mix after they have been sprayed onto the heating element 124 for vaporization.

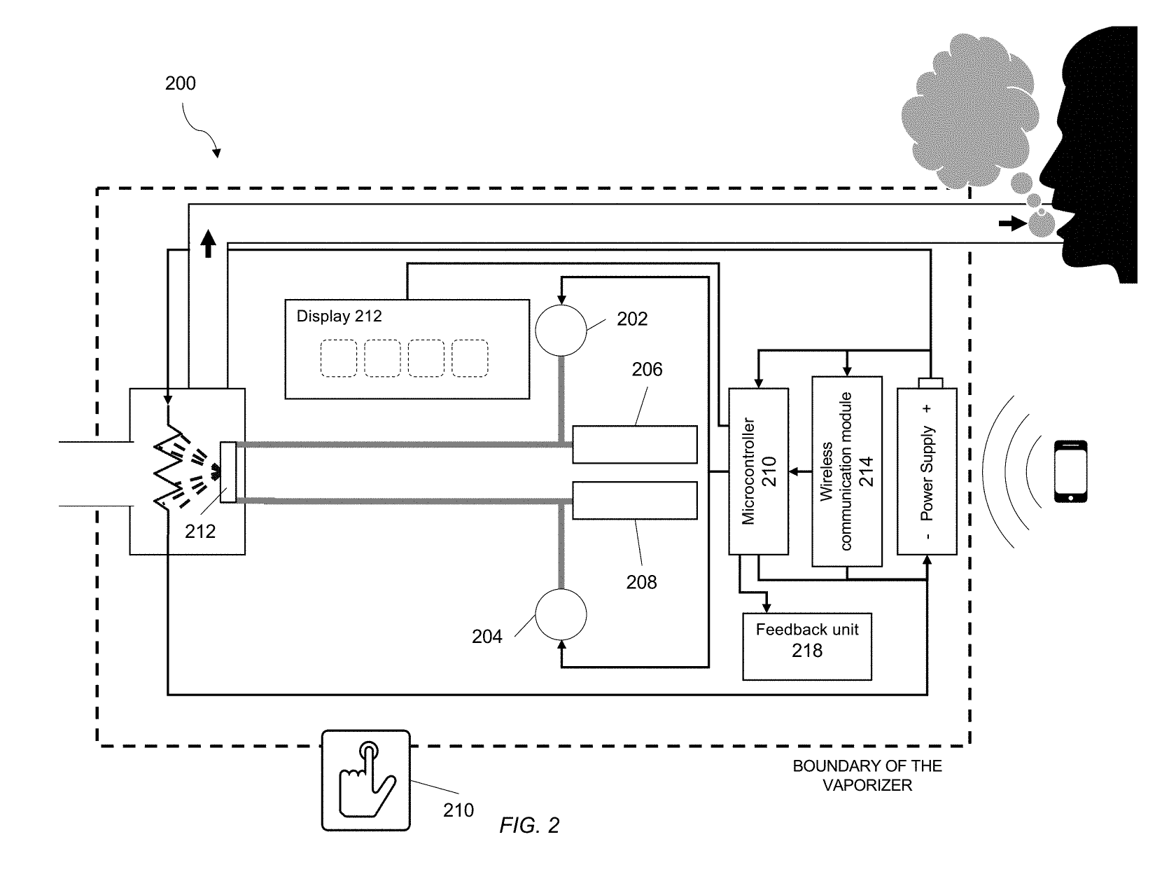

In some embodiments, instead of an acoustic source to motivate fluid flow into microfluidic channels, systems incorporating pumps can be implemented. FIG. 2 shows a schematic of a vaporizer 200 having several pressure-generating pumps 202 & 204 that are used to motivate fluid to leave corresponding liquid cartridges 206 & 208. Many components in the example shown in FIG. 2 are similar, the same as, or analogous to components in the example shown in FIG. 1, and where a component is not explicitly described with respect to FIG. 2, a description of a similar, same, or analogous component in FIG. 1 should be interpreted as applying to the component shown in FIG. 2. In some embodiments, the pressure-generating pumps 202 & 204 create positive pressure that pushes fluid out of the liquid cartridges 206 & 208, while in other embodiments, the pressure-generating pumps 202 & 204 create negative pressure to pull fluid out of the liquid cartridges. The end result is the same: vaping liquids are drawn out of the cartridges 206 & 208 in a controlled way to bring about a desired vaping result (e.g., a specific ratio of liquids, a desired quantity of liquids, or both).

The pumps 202 & 204 shown in FIG. 2 can be controlled by the microcontroller 210 such that quantity of liquid leaving each liquid cartridge 206 & 208 can be carefully controlled, resulting in a precise mixture of liquids from each of the liquid cartridges 206 & 208. Each pump 202 & 204 can have an air intake to facilitate creating pressure to motivate liquid out of the cartridges 206 & 208. The amount of pressure to move liquid out of a cartridge depends on several factors including the viscosity of the liquid contained the cartridge and the shape and size of the cartridge. It is contemplated that the microcontroller 210 can account for fluids having different fluid properties (e.g., viscosity) by altering behavior of a pump (e.g., pressure can be applied constantly or variably based on different fluid properties of the liquids contained in the liquid cartridges).

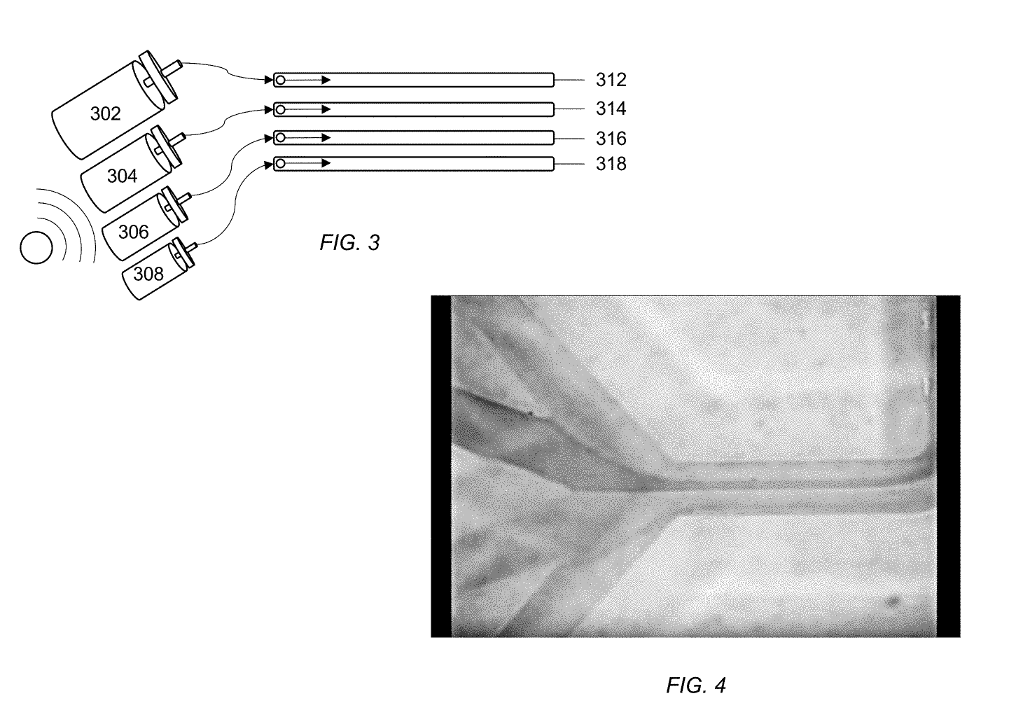

Whether implementing actuators as shown in FIG. 1 or pumps as shown in FIG. 2, liquid flow from a cartridge can be controlled by the microcontroller. A microcontroller in a vaporizer of the inventive subject matter controls the actuators 114 & 116 or the pumps 202 & 204 (e.g., it can affect frequency or amplitude of compression waves generated by actuators 114 & 116, or it can control a magnitude of pressure created by pumps 202 & 204). For example, volumetric flow rate of fluid from a cartridge can be affected by changing an amplitude or a frequency of the mechanical energy (e.g., compression waves) emitted from an actuator (e.g., either of actuators 114 or 116). As shown in FIG. 3, vaping liquid can be contained in resonance cavities 302, 304, 306, & 308. An actuator 310 (e.g., a compression wave generator of any kind) can then generate an output that causes fluid to move out of the resonance cavities 302, 304, 306, & 308 and into corresponding microfluidic channels 312, 314, 316, & 318. Volumetric flow rate of liquid from each resonance cavity depends, at least in part, on the frequency and amplitude of the compression waves generated by the actuator 310, as well as the cross-sectional shape and cross-sectional area of the microfluidic channel coupled with each resonance cavity and the material that each microfluidic channel is made from. Cartridges can include such resonance cavities containing vaping liquid.

Because the microcontroller in some embodiments is responsible for controlling actuators or pumps (depending on the embodiment of the system), volumetric flow rate of vaping liquid through microfluidic channels is controlled by the microcontroller. As discussed above, the microcontroller can change the amplitude and frequency of each acoustic source or actuator's output, depending on, for example, a desired mixture or quantity of liquid (e.g., ratios of each liquid) to be vaporized and inhaled. In embodiments with pumps, the microcontroller can cause the pumps to produce a desired amount of positive or negative pressure, depending on the requirements and internal configuration of a vaporizer system.

As mentioned above, a liquid contained in a resonance cavity (e.g., within a cartridge) can be motivated to leave the cavity by mechanical energy in the form of compression waves generated by an actuator (e.g., an acoustic source such as a transducer). As shown in FIG. 3 different resonance cavities respond to acoustic energy differently. Resonance frequency of a particular resonance cavity depend not only on the size and configuration of the resonance cavity, but also on the material properties of the liquid contained within it. When acoustic energy is applied to a resonance cavity at a resonance frequency (e.g., 390-410 Hz for resonance cavity 302, 470-490 Hz for resonance cavity 304, 515-545 Hz for resonance cavity 306, or 640-660 Hz for resonance cavity 308), the liquid contained within the resonance cavity leaves the cavity most easily (e.g., resulting in the highest volumetric flow rates through a corresponding microfluidic channel). For example, when amplitude of acoustic energy at a resonance frequency being applied to a resonance cavity is increased, volumetric flow rate of the fluid leaving that resonance cavity increases.

Once fluid leaves a resonance cavity (which is contained within a cartridge), it moves through a microfluidic channel coupled with that resonance cavity. In FIG. 1, for example, liquids from cartridges 102 & 104 move through microfluidic channels 118 & 120 toward the combining chamber 122. In embodiments with multiple cartridges (and thus multiple resonance cavities), the liquids from each resonance cavity can be combined into a common microfluidic channel as shown in FIG. 4 prior to atomization and vaporization. Liquids from two or more resonance cavities can be combined in specific ratios according to, for example, volumetric flow rates from each of the resonance cavities. Vaping liquid characteristics such as density, viscosity, resonance frequency, etc. can be useful in determining an appropriate actuator (e.g., an air pump, a transducer, a wick, etc.) for use in dispensing the vaping liquid. In some embodiments, the liquids are mixed prior to vaporization, while in other embodiments, the liquids are vaporized as they arrive at the heating element, where mixing is brought about via user inhalation.

In embodiments using, for example, transducers to motivate liquid to flow out of a cartridge, a transducer causes the cartridge containing the vaping liquid to vibrate at a resonance frequency (e.g., of the liquid, of the resonance cavity, or of both together) to control the amount of liquid that is dispensed from the cartridge. Resonance frequency of, for example, a vaping liquid is a function of the speed of sound through the liquid which in turn is a function of the viscosity and density of the liquid. As the resonance frequency of the vaping liquid increases, the requirements of a transducer used to vibrate a cartridge at a liquid's resonant frequency can also change. For example, e-juice, which is primarily composed of propylene glycol (PG) or vegetable glycerin (VG), has a resonance frequency on the order of 10s to 100s of KHz, whereas the least viscous cannabis oil extract used in vaporizers today are speculated to have a resonance frequency on the order of 10s to 100s of MHz. Compression waves that can match these different resonance frequencies can be generated using different components. Some frequencies (e.g., on the order of 10s to 100s of KHz) can be generated using, for example, miniature speakers whereas other frequencies (e.g., on the order of 10s to 100ss of MHz) can be generated using, for example, an ultrasonic transducer (e.g., a piezoelectric transducer).

In embodiments implementing air pumps (e.g., the embodiment shown in FIG. 2), air pumps can be selected based on, for example, viscosity and inertia of fluids to be motivated out of the cartridges. Air pump selection can also depend on channel cross-sectional area and other related factors. Air pumps must be strong enough to cause the fluids to flow through channels at desired volumetric flow rates for later mixing and vaporization. In most instances, vaping liquid to be used with systems of the inventive subject matter is similar in fluid properties to the e-juice commonly used in e-cigarettes, and can be made to include nicotine, cannabinoids, and flavoring. Air pump embodiments can implement microfluidic channels, or they can implement ordinary channels for liquid movement. An ordinary channel can facilitate liquid movement without the need for the generation of compression waves at any particular frequency, resonant or otherwise.

Cartridges of the inventive subject matter can be disposable or re-usable. A cartridge can include a fixed amount of liquid and is made from materials (e.g., hard plastic, aluminum, glass, etc.) that can be vibrated at a resonant frequency of a vaping liquid, of a cartridge, or of a cartridge and vaping liquid together. In some embodiments, a cartridge can have a standard interface for coupling with a vaporizer (e.g., a 510 interface, or any other cartridge/vaporizer interface, standardized or otherwise). In some embodiments, cartridges do not include a wick or heating coil, while in other embodiments, cartridges can include both of these components. For example, in microfluidic embodiments, cartridges do not need to include the wick or heating coil, while in time-controlled embodiments, cartridges can include both these components. Time-controlled embodiments are discussed below.

As mentioned above, FIG. 4 shows liquids in microfluidic channels combining in a single microfluidic channel at a specific ratio. That ratio, as described above, is achieved via the microcontroller, which controls volumetric flow from each acoustic source. Both the ratio of the liquids and the speed at which the liquids enter the common channel can be controlled by the microcontroller.

The process of combining liquids from resonance cavities (e.g., cartridges) can be carried out in a mixing chamber. In embodiments implementing microfluidics, the mixing chamber can include a common microfluidic channel like the one shown in FIG. 4. FIGS. 1 & 2 show example vaporizers having a combining chamber 122 & 212. Turning to FIG. 1, with liquids combined in the combining chamber 122, a heating element 124 (e.g., an energized coil, a metallic mesh, or the like) heats the mixture of liquids until a point of vaporization, at which point the vaporized liquids can be consumed by inhalation. While the heating element 124 is activated, a user inhales through the inhalation port 126. The heating element 124 can be controlled by the microcontroller (e.g., to control temperature and duration of energization). To create an inhalable vapor, the heating element 124 is contained within a vaporizing chamber 128 where it can vaporize the combined fluid mixture from the liquid cartridges 102 & 104 and also incorporate ambient air to ensure the vaporized fluids are not so harsh. Ambient air is brought in from an air intake 130, and, in some embodiments, an amount of ambient air that can be incorporated can be controlled by the microcontroller (e.g., by adjusting the aperture of an inlet for ambient air). In some embodiments, air intake 130 is a fixed component that allows air in when a user inhales.

The vaporizers shown in FIGS. 1 & 2 area configured to vaporize a specific amount, a specific ratio, or both, of vaping liquids. Because of this, using a wick system is impractical for these embodiments (i.e., embodiments that implement microfluidics). Wicks are typically designed to be consistently wet with vaping liquid for continuity of vaping. Vaporizer 100, on the other hand, is designed to allow a user to customize their vaping experience for each inhalation by controlling a quantity of vaping liquid(s) that is vaporized. For this reason, an atomizer of systems of the inventive subject matter can be included that operates similar to a wick-less Rebuildable Dripping Atomizer (RDA). The atomizer can atomize combined vaping liquid from the mixing chamber so that it comes into contact with the heating element during each use (e.g., during each inhalation by a user).

Methods of the inventive subject matter that implement microfluidics are also contemplated. In some embodiments, methods include all or any subset of the steps of: receiving user information including one or any combination of a user's medical history, current state of health, weight, BMI, genetic information, symptoms, and so on; identifying at least one recommended cartridge based on the user's medical information; receiving, in a vaporizing device, the at least one recommended cartridge; using an acoustic source to move fluid from the at least one cartridge into a microfluidic channel at a pre-determined fluid flowrate, where the pre-determined fluid flow rate is based on the user's medical history; vaporizing the liquid for inhalation.

In some embodiments, methods can further include adjusting the temperature of the heating element that vaporizes the fluid. It is contemplated that additional liquid cartridges and accompanying acoustic sources can be included to accommodate different combinations of liquids. For example, in embodiments that include two or more liquid cartridges, fluid flow of the liquids from those cartridges can be carefully controlled to administer a precise dose of vaporized liquid (e.g., 30% of a first liquid and 70% of a second liquid, where the first liquid is THC and the second liquid is CBD).

It is contemplated that some embodiments can include one or more heating elements. In some embodiments, there is a heating element for each cartridge, enabling a system to vaporize each vaping liquid independently. In other embodiments, there is a single heating element so that liquid from each cartridge is carried to the single heating element for simultaneous vaporization.

The heating element can include one or more metal meshes that are energized to vaporize vaping liquid. In some embodiments, the metal mesh is formed into a cylinder. The metal mesh can be made from titanium or nickel, and it can then be coupled with one or more of the microfluidic channels. For example, in vaporizer 100, the heating element 124 can include one or two metal meshes. In embodiments with a single heating element, liquid from both microfluidic channels are vaporized by the same heating element, while in embodiments with multiple heating elements, each microfluidic channel can lead to a separate heating element for vaporization. Vaping liquid moving from microfluidic channels can be deposited onto a heating element by capillary forces. When the heating element is energized, the one or more metal meshes are heated up and vaping liquid is vaporized for inhalation.

To activate a system of the inventive subject matter, some embodiments include a button (e.g., a physical push-button or a button that is activated by touch). As shown in FIGS. 1-3, the button (132, 210, or 520) can be pressed when a user is ready to inhale vaporized vaping liquid. In the vaporizer in FIG. 1, for example, when the button 132 is pressed, the heating element 124 is energized, and the actuators 114 & 116 drive a pre-specified amount of each vaping liquid through the microfluidic channels 118 & 120 to the mixing chamber 122. From there, the mixed liquids can be atomized (or otherwise deposited onto the heating element 124) and then vaporized by the heating element 124 for inhalation. Pressing the button, in some embodiments, causes a vaporizer to operate according to operating parameters that are set according to user preference, default settings, cartridge contents, and so on, while in other embodiments, the button acts as an on/off switch.

It is also contemplated that the act of inhalation can cause the above-described process to take place. In some embodiments, when a user inhales, vaporization takes place. In such embodiments, the button can be used to adjust certain parameters like temperature of the heating element, a quantity of vaping liquid to vaporize, a duration that the heating element is activated for, and so on.

In some embodiments, software can be run on an external computing device (e.g., a smartphone), where that software is designed to allow a user to control or adjust a vaporizer via the software running on the computing device. It is contemplated that some embodiments of vaporizers of the inventive subject matter can include a screen (e.g., display 134 & 212) and sufficient inputs such that a separate computing device is unnecessary. In the displays shown in FIGS. 1-3, the inputs are the displays themselves (e.g., a resistive or capacitive touch screen).

Software can be configured to allow users to, for example, customize the ratio of two or more vaping liquids based on personal preference. In embodiments where the software runs on an external device (e.g., a cell phone or computer), the external device can connect with a system of the inventive subject matter by a wired (e.g., USB) or wireless connection (e.g., Bluetooth, WiFi, etc.), where the wireless connection is facilitated by a wireless communication module (e.g., wireless communication modules 108 & 214 in FIGS. 1 & 2).

Within the software, a user can select, for example, an amount of vaping liquid to be dispensed from each cartridge within a system. Liquid quantity can be measured by, for example, volume (e.g. 0.8 mL of strawberry-flavored nicotine-based vaping liquid and 0.6 mL of CBD-based vaping liquid) or the dosage of specific molecule or compound to be consumed (e.g., 1 mg of THC, or 3 mg of CBD, or some combination thereof in some ratio). Users can also adjust vaping temperature as needed or desired.

Some embodiments of the inventive subject matter include an accompanying software application that can be installed on a computer device (e.g., a smartphone). The software can be used to gather personal and health-related information from the user, such as weight, height, medical history, smoking (e.g., cigarettes) history, vaping history, the reason why the user is using the vaporizer (e.g., a user's vaping goal), etc.

The software can then use collected information to make recommendations to the user about, for example, the vaping liquids they can use to meet their vaping goal. For example, the software can recommend different ratios of THC, CBN, and CBD, total quantity of a particular mixture to be vaporized, etc. Once the software has developed a recommendation, the software can then facilitate purchasing of the necessary cartridges (e.g., by giving a user links to purchase cartridges (e.g., cartridges that would be needed to create the recommended mixture).

Cartridges can include an identifier that can be used to get more information about the cartridge or information related to the cartridge. For example, a cartridge can include one or any combination of a QR code, an RFID tag, a barcode, etc. Once a user receives a cartridge, they can use the identifier to gather information about the cartridge (e.g., by scanning or otherwise reading its information) before plugging the cartridge into the vaporizer. Once information about a cartridge gathered, the software can recognize the cartridge and load information about the cartridge from a database (e.g., density, viscosity, resonance frequency, etc.).

The vaporizer can then receive information from the software regarding, for example, how to vaporize each vaping liquid, as well as how much of each vaping liquid to dispense to deliver a pre-specified dose of each liquid (which can be, e.g., determined in by software). A pre-specified dose can be initially provided by the software algorithmically, considering, for example, personal and health-related information about a user as well as the user's vaping goal.

A user can thus use a vaporizer of the inventive subject matter while at the same time using both a screen on the vaporizer (in embodiments where a screen is included) and while using the software on a mobile device to monitor intake of vaping liquids. The software can regularly (e.g., daily) collect user information related to effectiveness of the user's vaping experience. For example, a user can be asked probing questions about how a user has enjoyed their experience: “On a scale of 1-5, how well is the 0.2 mg Sativa vaping liquid with 0.5 mg Coffee-Flavored vaping liquid mix helping you overcome your morning depression?” Of course, users can opt out of providing this information or allowing this information to be shared.

In some embodiments, user information can be used in a wide variety of ways. User information can be transmitted from vaporizer to computing device to servers where the information can be used to improve the vaping experience of all users. User information can be transmitted periodically (e.g., on an hourly, daily, weekly, or other periodic basis). User information can then be used to determine the effectiveness of the initially suggested dosage and help customize the user's vaping experience. In some embodiments, users can pay for a virtual doctor visit that allows a user to, for example, video chat with a doctor to ask questions about their customized prescription or vaping mixture, review vaping history to see whether it has been effective for the user, etc. User data can be used by data scientists to, for example, track effects of consumption of various vaping liquid mixtures to improve future recommendations.

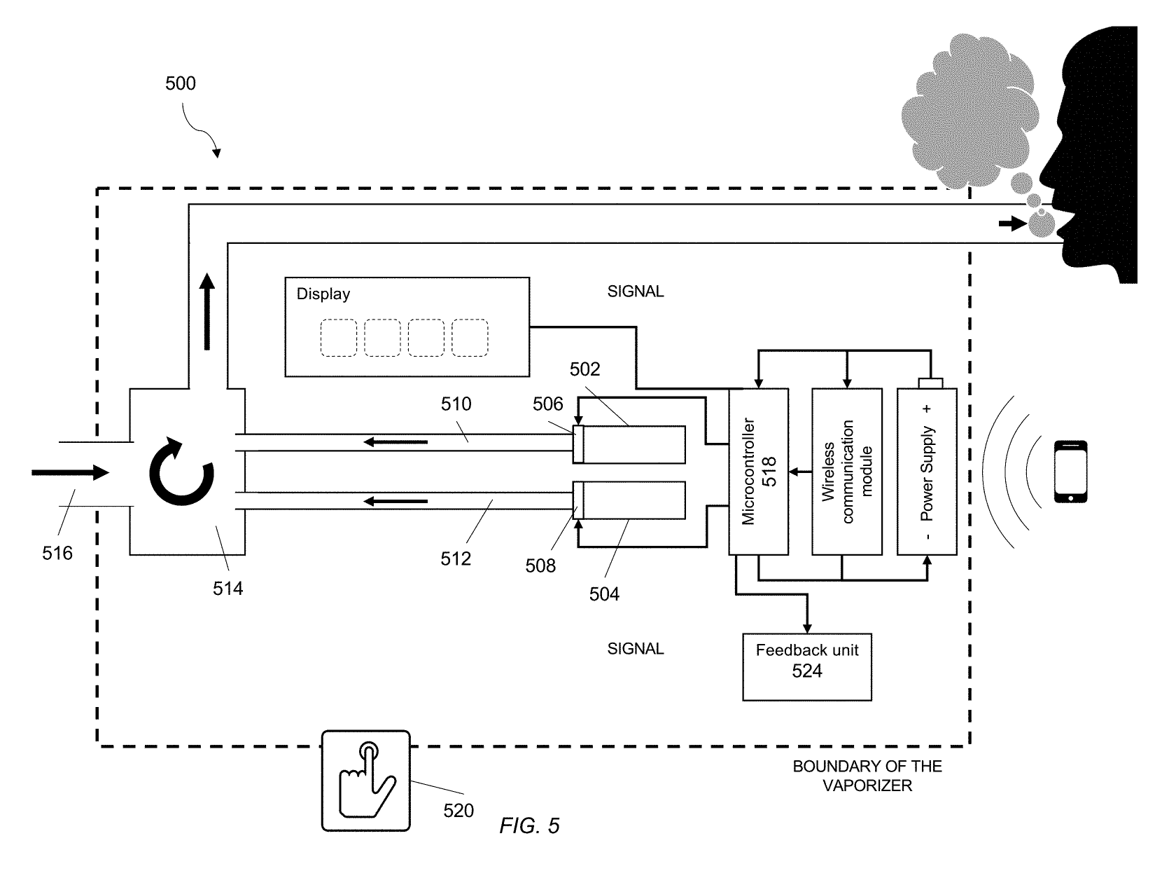

Time-controlled systems for vaporizing vaping liquids are also contemplated. A time-controlled system relies on energizing a heating element for a specific amount of time, either with or without accurately accounting for how much vaping liquid is vaporized during that time. For example, the examples shown in FIGS. 1 & 2 can vaporize a specific amount and specific ratio of vaping liquid. The vaporizer 500 shown in FIG. 5, on the other hand, is configured to vaporize vaping liquid for a specific amount of time, where the amount of time can be based on, for example, a desired amount of liquid to be vaporized and how long it takes on average for that much liquid to be vaporized.

Vaporizer 500 can include two or more cartridges 502 & 504, and the cartridges that are used with vaporizer 500 can include vaporizing elements 506 & 508. Vaporizing elements 506 & 508 can include, for example, both a wicking element and heating element, which enables the vaporizing elements 506 & 508 to produce vaporized vaping liquid for later inhalation. Vaporized vaping liquid travels through channels 510 & 512 into a mixing chamber 514 where it is mixed with ambient air introduced through an inlet 516 and then inhaled by a user.

Vaporizing elements 506 & 508 are activated by the microcontroller 518, causing vaping liquid to be vaporized. Time controlled embodiments are capable of vaporizing mixtures of vaping liquids from multiple cartridges. For example, if a user wants to inhale a mixture of 30% of a vaporized liquid from cartridge 502 with 70% of liquid from cartridge 504, then the microprocessor can cause vaporization of the liquid from cartridge 502 to occur for 30% of some total time and it can cause vaporization of the liquid from cartridge 504 for 70% of some total time (e.g., cartridge 502 is energized for 0.3 seconds and cartridge 504 is energized for 0.7 seconds, or any other combination of times resulting in a desired ratio of vaporized liquid). The total time for inhalation can be user-defined. For example, a user can input that they want to inhale for two seconds. To achieve a desired ratio of liquids during that two seconds, the cartridge that requires longer vaporization can be energized for the entire two seconds, while the cartridge that requires less time can be energized only for an amount of time necessary to bring about a desired mixture.

In some embodiments, the cartridge that is energized for a shorter duration can be energized continuously, or it can be energized in pulses that stretch out its energization for the entire time that the longer duration cartridge is activated for. Activation of the cartridges in a vaporizer of the inventive subject matter can thus be done using one or more pulse-width modulators. This is analogous to using a pulse-width modulator to activate a motor: a pulse-width modulator can cause a motor to turn slower than a maximum speed by only energizing it for 30% of a total time (e.g., a 30% duty cycle) via electrical pulses delivered at some frequency that causes the motor to turn smoothly. Frequency of the pulses (e.g., the switching frequency) that can cause adequate vaporization can range from low frequencies, for example, 3 Hz to 10 Hz, or, in some embodiments, higher frequencies, for example, 10 Hz-1 kHz. Thus, controlling how long each cartridge is energized for in a multi-cartridge system can result in any desired ratio of vaporized liquids for a user to inhale.

In some embodiments, both vaporizing elements 506 & 508 are activated according to pulse-width modulation. When both vaporizing elements 506 & 508 are activated according to pulse-width modulation, vaporizers of the inventive subject matter can make it easier for users to inhale more comfortably. When users inhale vaporized vaping liquid without any air mixed in, for example, it would be very difficult to inhale without coughing. Thus, many vaporizers, including the one shown in FIG. 5, include an air inlet to mix air in with the vaporized vaping liquids. By activating both vaporizing elements 506 & 508 according to a duty cycle and pulse frequency (e.g., activating by a pulse-width modulator), the mixture that a user inhales can thus include more air over the course of an inhalation than if that user were to inhale vaping liquid that is vaporized without pulse-width modulation. For example, if a vaporizing element is activated according to a 50% duty cycle, then that user would inhale much more air over a given period of time than if the vaporizing element were to operate at 100% capacity over the same given period of time.

As with other embodiments described in this application, vaporizer 500 can be equipped with a wireless communication module 520. The wireless communication module can thus be configured to allow the vaporizer 500 to communicated with external computing devices, such as cell phones and tablets. As discussed above, software can be installed on an external computing device that can be used to change different settings in the system so that a user can achieve a desired vaping experience (e.g., a desired ratio of vaping liquids).

Vaporizers like the one in FIG. 5 can also be configured to delivery desired quantities. When the rate at which liquid is vaporized from a particular cartridge is known, then quantity can be controlled in a number of ways. For example, if it is known that a cartridge delivers 1 mg of THC per second, and a user wants to inhale 2 mg from that cartridge, then the system can be configured to vaporize liquid from that cartridge for two seconds. If, on the other hand, the user wants to inhale for two seconds but only wants to receive 1 mg of THC, then the system can cause that cartridge to be active only for one second for the total 2 seconds of inhaling. This effect can be achieved using, for example, a pulse-width modulator set to 50%.

As with other embodiments, a button 520 can be included to activate the vaporizer 500. In some embodiments, the vaporizer 500 is activated by inhalation instead of a button press. It is contemplated that the button 520 can be held down and, upon having the vaporizer 500 activated for a pre-specified amount of time, the vaporizer can give a user an alert via, for example, a display 522 (in embodiments where a display is included). An alert can also be delivered via a feedback unit 524 in the form of any one or combination of a sound, a vibration, or light.

Systems and methods of the inventive subject matter improve mixing of various cannabinoid-based vaping liquids. For example, users can choose ratios and amounts of CBD, THC, Sativa, Indica, and so on, to create a personalized entourage effect. The term entourage effect refers to a concept and proposed mechanism by which compounds present in cannabis which are largely non-psychoactive by themselves modulate the overall psychoactive effects of the plant (these resulting principally from the action of the main psychoactive component of cannabis, THC). Thus, various cannabinoid-based vaping liquids can be mixed with flavored vaping liquids to augment or eventuate the effect of cannabis consumption. For example, an Indica-based vaping liquid can be mixed with a lavender-flavored vaping liquid to create a relaxing effect, or a Sativa-based vaping liquid and mint-flavored vaping liquid can be mixed to create an uplifting effect.

As mentioned above, embodiments of the inventive subject matter can implement microfluidic physics to precisely regulate the flow of liquids in small doses, as shown in FIG. 1. Embodiments implementing microfluidics use acoustic compression waves generated by acoustic actuators to regulate the flow of the vaping liquid from cartridges. One parameter affecting the design of microfluidic systems is the nature of the vaping liquids contained within each cartridge. Vaping liquids should be of a chemical constituency that allows for inclusion of nicotine-based, cannabis-based, or pure flavoring-based compounds. A vaping liquid is likely to have a chemical constituency similar to the fluids used in e-cigarettes (“e-juices”). E-juices can include various amounts of nicotine and various flavoring. But cannabis-based vaping liquids are not as common, and they typically include CBD but not other cannabinoids like THC or CBN. It is contemplated that vaping liquids of the inventive subject matter can contain all types of cannabis-related chemical compounds.

In some embodiments, vaporizers of the inventive subject matter can alert a user once, for example, a pre-specified amount of vaping liquid has been vaporized, a pre-specified amount of time has elapsed since a vaporizer was activated (e.g., time elapsed since vaporized vaping liquid has been created for inhalation), or a pre-specified amount of time has elapsed since a user has begun to inhale. Alerts can be delivered via a feedback unit (e.g., feedback unit 132, 218, & 524). An alert can include one or any combination of sound (e.g., via a feedback unit having a speaker), vibration (e.g., via a feedback unit having a vibration mechanism), and light (e.g., via a display or a feedback unit having one or more light sources). If a user does not hold the activation button down long enough, a system can indicate that some amount (e.g., a percent) of the pre-specified amount of vaping liquid has been vaporized with some amount still remaining in, for example, a mixing channel. If the button is held after the device alerts a user that the pre-specified quantity of vaping liquid has been vaporized, the heating element can be de-energized so that no additional vaping liquid will be deposited onto the heating element for vaporization. In some embodiments, the device begins a second dose consecutively after the first, issuing a second alert once the second dose has been delivered.

Some embodiments include a screen that can be used to give a user information about the device (e.g., device parameters). The visible parameters can be set by adjusting the device's settings either directly on the device or via, for example, an external device that is configured to communicated with a device of the inventive subject matter. In some embodiments, external devices can be configured to execute software that is written to cause the devices to communicate with a vaporizer so that users can adjust settings and so forth.

Device parameters can include: a dosage (e.g., a default dosage or a user-defined, pre-specified dosage), a heating coil temperature (e.g., a temperature measurement in, for example, F or C, or a relative temperature indication such as low, medium, or high), a duration for inhalation (e.g., an amount of time the button can be held before an alert will be delivered), a percent or quantity of each vaping liquid to be vaporized during a button press.

Thus, specific systems and methods of electronic vaporizers have been disclosed. It should be apparent, however, to those skilled in the art that many more modifications besides those already described are possible without departing from the inventive concepts in this application. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A vaporizer, comprising:
a first cartridge connector configured to receive a first cartridge containing a first vaping liquid and configured to vaporize the first vaping liquid upon activation;
a second cartridge connector configured to receive a first cartridge containing a second vaping liquid and configured to vaporize the second vaping liquid upon activation;
a microcontroller configured to create a blend of vaporized vaping liquid, the blend comprising the first vaping liquid and the second vaping liquid, wherein the blend comprises a pre-specified ratio of the first vaping liquid to the second vaping liquid;
wherein upon coupling the first and second cartridges to the first and second cartridge connectors the microcontroller is configured to activate the first cartridge to vaporize the first vaping liquid for a first duration of time and to activate the second cartridge to vaporize the second vaping liquid for a second duration of time; and
wherein the first duration of time is different from the second duration of time, and the pre-specified ratio of the blend is correlated with a ratio of the first duration of time to the second duration of time.

2. The vaporizer of claim 1, wherein the ratio of the first duration of time to the second duration of time is approximately the same as the pre-specified ratio.

3. The vaporizer of claim 1, further comprising a display and at least one input

4. The vaporizer of claim 1, further comprising a wireless communication module to facilitate connection a mobile device, wherein the mobile device is configured to receive input from a user and transmit information based on the input to the vaporizer, the input comprising settings for the vaporizer.

5. The vaporizer of claim 4, wherein the settings comprise at least one of a first time duration and a second time duration

6. The vaporizer of claim 1, wherein the microcontroller is configured to activate the first cartridge using a pulse-width modulator that generates electrical pulses according to a duty cycle and a switching frequency, wherein the duty cycle results in the first cartridge being activated for the first duration of time, and wherein the first duration of time is distributed over the second duration of time according to the duty cycle and the switching frequency.

7. The vaporizer of claim 1, wherein the microcontroller is configured to:
activate the first cartridge using a first pulse-width modulator that generates electrical pulses according to a first duty cycle and a first switching frequency,
activate the second cartridge using a second pulse-width modulator that generates electrical pulses according to a second duty cycle and a second switching frequency, and
wherein the first and second cartridges are activated over a total time duration, wherein the total time duration is longer than both the first time duration and the second time duration.

8. The vaporizer of claim 7, wherein the first duty cycle is different from the second duty cycle.

9. The vaporizer of claim 1, wherein the first cartridge and the second cartridge are activated for at least some overlapping amount of time.
